(19) United States
(12) Patent Application Publication
(10) Pub. No.: US 2025/0345555 A1
(43) Pub. Date: Nov. 13, 2025

(54) METHOD AND APPARATUS FOR MANAGING PSYCHOLOGICAL STATE THROUGH INFLATABLE VEST WITH NON-CONTACT BIOSENSOR

(71) Applicant: DOLBOMDREAM CO. LTD., Daejeon (KR)

(72) Inventors: Jihun KIM, Goyang-si (KR); Seokhyun Kim, Daejeon (KR)

(73) Assignee: DOLBOMDREAM CO. LTD., Daejeon (KR)

(21) Appl. No.: 19/276,091

(22) Filed: Jul. 22, 2025

Related U.S. Application Data
(63) Continuation-in-part of application No. PCT/KR2024/095043, filed on Jan. 22, 2024.

(30) Foreign Application Priority Data
Jan. 31, 2023 (KR) 10-2023-0012525

(51) Int. Cl. A61M 21/02; A61B 5/1171; A61M 21/00; G16H 40/63; H02J 7/00

(52) U.S. Cl. CPC A61M 21/02; G16H 40/63; H02J 7/0044; A61B 5/1176; A61M 2021/0022; A61M 2205/07; A61M 2205/14; A61M 2205/3306; A61M 2205/3317; A61M 2205/3327; A61M 2205/3344; A61M 2205/3368; A61M 2205/3375; A61M 2205/609; A61M 2205/8206; A61M 2205/8237

(57) ABSTRACT

According to an embodiment of the present disclosure, a system for managing a psychological state of a user based on biometric information of the user includes a wearable device comprising an air tube configured to apply pressure to a portion of the user's body and an air pump configured to inject air into or discharge air from the air tube, and an external device configured to operate an artificial intelligence model, wherein the artificial intelligence model is configured to generate control information for controlling the air pump of the wearable device based on output data obtained by using the biometric information as input data, and is trained using the biometric information.

### CROSS REFERENCE TO PRIOR APPLICATIONS

This application is a continuation application of National Stage Application of PCT International Patent Application No. PCT/KR2024/095043 filed on Jan. 22, 2024, under 35 U.S.C. § 371, which claims priority to Korean Patent Application No. 10-2023-0012525 filed on Jan. 31, 2023, respectively, which are all hereby incorporated by reference in their entirety

### BACKGROUND OF THE INVENTION

The present disclosure relates to a method and an apparatus for managing a psychological state based on biometric information, and more particularly, to a method and an apparatus for providing psychological care to a user, such as a child or individual with a developmental disorder, by utilizing a wearable device including a biometric sensor. Biometric information of the user is obtained through the biometric sensor embedded in the wearable device, such as an air-inflatable vest, worn by the user, and based on the obtained biometric information, deep touch pressure (DTP) stimulation is applied to the user via one or more air tubes provided in the wearable device. The DTP stimulation may be performed in a manner corresponding to the user's physiological state as determined by the biometric information, thereby providing the user with an appropriate sense of psychological stability and enabling effective care of the user's psychological condition.

With the growing importance of managing and stabilizing a person's psychological state or stress in recent years, there is an increasing demand for methods and apparatuses capable of providing psychological care. In particular, as the population of individuals requiring psychological care—such as infants, children, adolescents, persons with disabilities, and the elderly—continues to grow and as the aging society progresses, the need for effective methods and apparatuses for managing psychological states or stress is becoming increasingly significant.

In this regard, various methods and technologies have been proposed for determining or estimating a user's psychological state or stress, in which biometric information related to the user's physiological condition is acquired and the user's psychological state is determined or estimated based on the acquired biometric information.

In addition, various methods and technologies have been proposed for stabilizing or managing a user's psychological state or relieving stress based on the user's psychological condition or stress level.

### SUMMARY OF THE INVENTION

In order to acquire the biometric information necessary for determining or estimating a user's psychological state or stress, a biometric sensor (e.g., a biometric sensor of a smartwatch) may acquire the user's biometric information while being in contact with the user's body. That is, since the biometric information is obtained while the sensor remains in contact with the user's body, the user may experience discomfort, a foreign body sensation, or a feeling of constraint due to the physical contact of the biometric sensor. Accordingly, there is a need for methods and technologies capable of acquiring a user's biometric information without making physical contact with the user's body, so as to avoid causing such discomfort.

Methods and technologies have been proposed for acquiring a user's biometric information from a distance without physically contacting or restraining the user's body. For example, biometric information may be remotely acquired using a biometric sensor such as a radar sensor. However, in order to acquire biometric information from a distance, the biometric sensor may need to be installed in a specific space or location, and biometric information may only be acquired when the user is present within the space where the sensor is installed. As a result, the user may experience spatial constraints. Accordingly, there is a need for methods and technologies capable of acquiring biometric information without physical contact with the user's body and without imposing spatial constraints on the user.

Various methods and technologies have been proposed for managing or stabilizing a user's psychological state or stress by providing visual or auditory content, such as videos or music, that induces psychological comfort. However, in order for a user requiring psychological care to utilize such methods, the user must subjectively determine when psychological stabilization is needed and manually select and play the appropriate video or music content. As a result, the assessment of the user's psychological state or stress may lack objectivity, and the process of manually selecting content may be inconvenient for the user. Accordingly, there is a need for methods and technologies capable of automatically determining the user's psychological state or stress level and providing an appropriate psychological calming stimulus (e.g., deep touch pressure) to the user based on the determined state, thereby allowing the user to objectively and conveniently manage their psychological condition.

Various embodiments disclosed herein may provide methods and apparatuses for addressing the aforementioned issues.

According to an embodiment of the present disclosure, a system for managing a psychological state of a user based on biometric information of the user may include a wearable device configured to apply pressure to a portion of the user's body, the wearable device comprising one or more air tubes for applying pressure and an air pump for injecting air into or discharging air from the air tubes, and an external device configured to operate an artificial intelligence (AI) model that generates control information for controlling the air pump of the wearable device based on output data obtained by using the user's biometric information as input data to the AI model, wherein the AI model is trained using the biometric information. The wearable device may include a first biometric sensor disposed at a front portion of the wearable device and configured to acquire the user's biometric information in a non-contact manner and to process the acquired biometric information via a biometric information processing module, a second biometric sensor disposed at a rear portion of the wearable device and configured to acquire the user's biometric information in a non-contact manner, a first communication module operatively connected to the first biometric sensor, a second communication module operatively connected to the second biometric sensor, a third communication module, and a processor electrically connected to the first and second biometric sensors, the air pump, and the third communication module. The processor may be configured to acquire first information regarding a first region of the user's body via the first biometric sensor, acquire second information regarding the same region via the second biometric sensor, determine overlapped information between the first and second information as first biometric information of the first region via the biometric information processing module, remove noise signals from the first biometric information to generate second biometric information via the biometric information processing module, and transmit the second biometric information to the external device via the first communication module.

According to an embodiment of the present disclosure, by acquiring the user's biometric information through a non-contact biometric sensor included in a wearable device (e.g., an air-inflatable pressure vest), the biometric information may be obtained without physical contact with the user's body, thereby eliminating the discomfort that may be caused by contact with the biometric sensor while still enabling effective acquisition of the biometric information.

According to an embodiment of the present disclosure, by acquiring the user's biometric information through a non-contact biometric sensor included in a wearable device (e.g., an air-inflatable pressure vest), the biometric information may be obtained without requiring the user to visit a specific space or location where the non-contact biometric sensor is installed, thereby enabling acquisition of the user's biometric information without spatial constraints.

According to an embodiment of the present disclosure, by acquiring the user's biometric information through non-contact biometric sensors respectively installed at the front and rear portions of a wearable device (e.g., an air-inflatable pressure vest), the accuracy of the biometric information may be enhanced.

According to an embodiment of the present disclosure, the psychological state or stress level of a user may be accurately determined in real time through an artificial intelligence model trained using not only biometric information acquired via contact or non-contact biometric sensors included in a wearable device (e.g., an air-inflatable pressure vest), but also at least one of various types of auxiliary data including user information (e.g., age, height, weight, gender, degree of developmental disorder), user movement information, user vision information, user location information, ambient noise information, ambient brightness information, weather information, or survey data provided by a guardian (e.g., psychological condition of the user).

According to an embodiment of the present disclosure, the optimal air pressure pattern to be applied to the user may be accurately determined in real time based on at least one of the following: biometric information acquired through contact or non-contact biometric sensors included in a wearable device (e.g., an air-inflatable pressure vest), the user's psychological state or stress level as determined by an artificial intelligence model, user information (e.g., age, height, weight, gender, degree of developmental disorder), user movement information, user vision information, user location information, ambient noise information, ambient brightness information, weather information, air pressure pattern information, or user response information (e.g., changes in the user's biometric data following air pressure application). According to another embodiment of the present disclosure, a heat dissipation effect may be provided by appropriately regulating the body temperature of the user wearing the wearable device through a blower and a plurality of ventilation holes formed in the wearable device (e.g., an air-inflatable device).

In addition, various effects that are directly or indirectly derived from the present disclosure may also be provided.

### BRIEF DESCRIPTIONS OF THE DRAWINGS

FIG. 1 illustrates a system for managing a user's psychological state based on biometric information, according to an embodiment of the present disclosure.

FIG. 2 illustrates a block diagram of a wearable device, according to an embodiment of the present disclosure.

FIG. 3 illustrates a block diagram of an external device, according to an embodiment of the present disclosure.

FIG. 4 illustrates a block diagram of an electronic device, according to an embodiment of the present disclosure.

FIG. 5 illustrates a flowchart of a method for acquiring biometric information of a user by a wearable device, according to an embodiment of the present disclosure.

FIG. 6 illustrates a flowchart of a method for removing noise from biometric information based on user movement information by the wearable device, according to an embodiment of the present disclosure.

FIG. 7 illustrates a flowchart of a method for switching a sensing mode of a biometric sensor based on attachment or detachment from the wearable device, according to an embodiment of the present disclosure.

FIG. 8 illustrates a flowchart of a method for sensing by a biometric sensor based on the number of sensors detached from the wearable device, according to an embodiment of the present disclosure.

FIG. 9 illustrates a flowchart of a method for sensing by a biometric sensor based on the number of sensors placed on a charging cradle after being detached from the wearable device, according to an embodiment of the present disclosure.

FIG. 10 illustrates a flowchart of a method for transmitting training data and input data of an AI model to an external device operating the AI model for determining a psychological state, according to an embodiment of the present disclosure.

FIG. 11 illustrates another flowchart of a method for transmitting training data and input data of an AI model to an external device operating the AI model for determining a psychological state, according to an embodiment of the present disclosure.

FIG. 12 illustrates a flowchart of a method for operating a blower of the wearable device, according to an embodiment of the present disclosure.

FIG. 13 illustrates a flowchart of a method for transmitting a first control signal from the external device to the wearable device to control the air pump for injecting air into the air tube, according to an embodiment of the present disclosure.

FIG. 14 illustrates a flowchart of a method for transmitting a second control signal from the external device to the wearable device to control the air pump for removing air from the air tube, according to an embodiment of the present disclosure.

FIG. 15 illustrates a flowchart of a method for determining a user's psychological state using a first artificial intelligence model by the external device, according to an embodiment of the present disclosure.

FIG. 16 illustrates a flowchart of a method for transmitting a first control signal from the external device to the wearable device to control the air pump for injecting air based on an air pressure pattern determined using a first and a second AI model, according to an embodiment of the present disclosure.

FIG. 17 illustrates a flowchart of a method for transmitting a second control signal from the external device to the wearable device to control the air pump for removing air based on the user's psychological state determined using a first AI model, according to an embodiment of the present disclosure.

FIG. 18 illustrates a flowchart of a method for controlling the air pump to inject air into the air tube by the wearable device based on the first control signal, according to an embodiment of the present disclosure.

FIG. 19 illustrates a flowchart of a method for controlling the air pump to remove air from the air tube by the wearable device based on the second control signal, according to an embodiment of the present disclosure.

FIG. 20 illustrates a flowchart of a method for displaying various types of information by an electronic device, according to an embodiment of the present disclosure.

In connection with the description of the drawings, identical or similar reference numerals may be used to denote identical or similar components.

### DETAILED DESCRIPTION OF THE INVENTION

Hereinafter, various embodiments of the present invention will be described with reference to the accompanying drawings. However, this is not intended to limit the present invention to the specific embodiments disclosed, and it is to be understood that the present invention encompasses various modifications, equivalents, and/or alternatives of the embodiments.

Hereinafter, embodiments of the present invention will be described in detail with reference to the accompanying drawings so that those skilled in the art to which the present invention pertains may readily carry out the invention. However, the present invention may be implemented in various different forms and is not limited to the embodiments described herein. In addition, parts irrelevant to the description have been omitted from the drawings in order to clearly illustrate the present invention, and like reference numerals are used to refer to similar elements throughout the specification.

Throughout the specification, when a part is described as “including” a certain component, this is to be understood as not excluding the presence of other components unless explicitly stated otherwise, but rather allowing the inclusion of additional components. In addition, terms such as “unit,” “device,” and “module” described in the specification refer to elements that perform at least one function or operation, and may be implemented as hardware, software, or a combination of hardware and software.

Throughout the specification, when a certain part is described as being “connected” to another part, it is to be understood as including not only cases where they are “directly connected” but also cases where they are “electrically connected” through one or more intervening elements. Furthermore, when a certain component is described as “including” another component, this is to be interpreted, unless specifically stated otherwise, as not excluding the presence of additional components. It should also be understood that the presence or addition of one or more other features, numbers, steps, operations, components, parts, or combinations thereof is not precluded in advance.

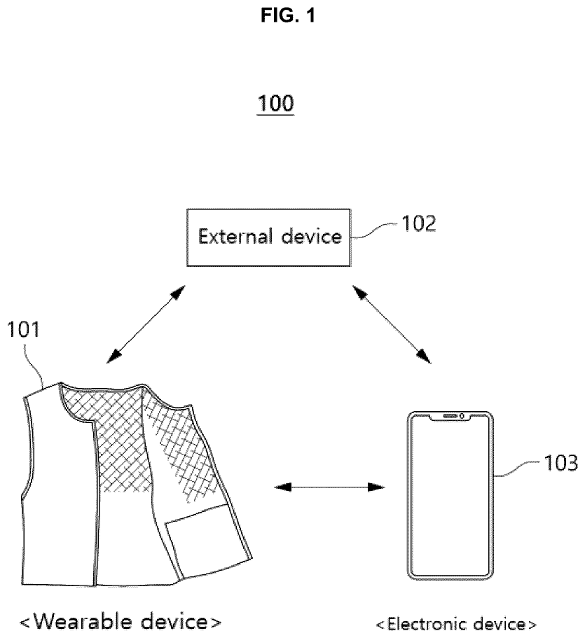

FIG. 1 illustrates a system (100) for managing a user's psychological state based on biometric information, according to an embodiment of the present disclosure.

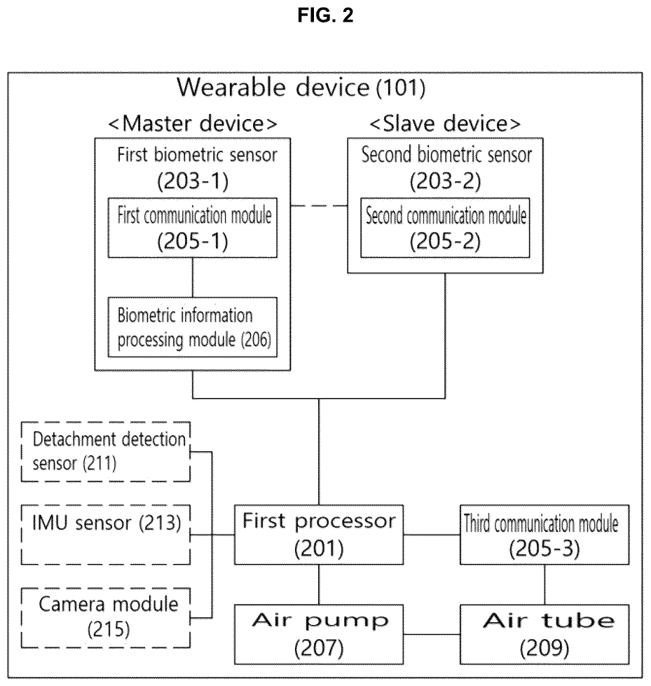

Referring to FIG. 1, a system (100) for managing a user's psychological state based on biometric information may include a wearable device (101), an external device (102), and an electronic device (103), and may be operated based on the wearable device (101), the external device (102), and the electronic device (103). However, the system (100) is not limited to the components illustrated in FIG. 1, and certain components may be omitted or additional components may be included. For example, the system (100) may further include a charging cradle configured to charge biometric sensors (e.g., the first biometric sensor (203-1) and the second biometric sensor (203-2) of FIG. 2) provided in the wearable device (101).

According to one embodiment, the wearable device (101) may be referred to as an air-inflatable pressure vest including one or more air tubes and an air pump for providing deep touch pressure (DTP) to the user. For example, the wearable device (101) may be understood as a means that can be worn by a user in need of psychological care and may include a wearable means configured to apply pressure (or force) to the user via pressure-generating components such as air tubes.

According to one embodiment, deep touch pressure refers to a type of pressure that stimulates the parasympathetic nervous system when an appropriate amount of pressure is applied to the human body, thereby giving the user a sensation similar to being hugged and inducing psychological comfort. The user may be a person wearing the air-inflatable pressure vest and may be a subject in need of psychological stabilization, such as a child or individual with a developmental disorder. However, the user is not limited to the examples described above and may refer to any person who requires psychological comfort. For example, the user may include an infant, child, adolescent, person with a disability, or elderly person.

According to one embodiment, the wearable device (101) may include one or more biometric sensors (e.g., the first biometric sensor (203-1) and the second biometric sensor (203-2) of FIG. 2) for acquiring biometric information of the user. The biometric sensor may include at least one of an electrodermal activity (EDA) sensor for measuring skin conductance, a photoplethysmograph (PPG) sensor, a sensor for measuring blood volume pulse (BVP), a sensor for measuring respiration (RESP), a thermal sensor for measuring heat or body temperature, a sensor for measuring heart rate variability (HRV), or a sensor for measuring heart rate (HR).

According to one embodiment, the wearable device (101) acquires biometric information of the user using the biometric sensor. The wearable device (101) transmits the biometric information acquired by the biometric sensor to at least one of the external device (102) or the electronic device (103) either in real time or at a designated interval. The wearable device (101) includes a plurality of biometric sensors, and each of the biometric sensors is individually equipped with a communication module. Each biometric sensor performs LTE communication independently using the communication module included therein. The plurality of biometric sensors provided in the wearable device (101) are collectively referred to as a sensor unit.

According to one embodiment, the wearable device (101) provides deep touch pressure to the user based on the biometric information. For example, the wearable device (101) controls the air pump to inject air into the air tube, thereby applying air pressure to the user and delivering deep touch pressure. The air tube, air pump, and the processor that controls the air pump, which are included in the wearable device (101), are collectively referred to as a driving unit.

According to one embodiment, the sensor unit and the driving unit of the wearable device (101) perform wireless communication (e.g., LTE communication) independently. The sensor unit and the driving unit support lifetime upgrade via wireless firmware over-the-air (FOTA) updates.

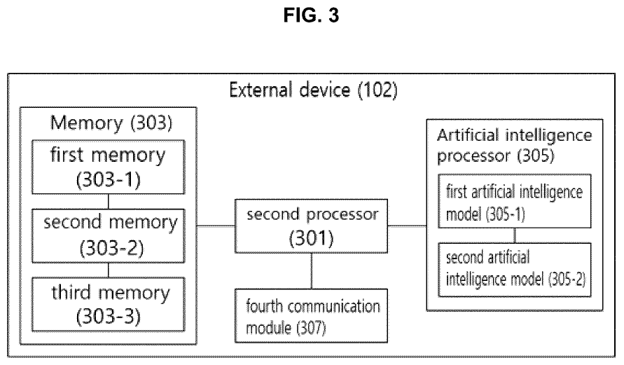

According to one embodiment, the external device (102) is referred to as a server that operates an artificial intelligence model (e.g., the first AI model (305-1) in FIG. 3) for determining the user's psychological state or stress level based on the user's biometric information. The external device (102) receives biometric information from the wearable device (101) in real time. The external device (102) inputs the received real-time biometric information into the AI model and determines the user's psychological state or stress level based on the output value generated by the model. The AI model (e.g., the first AI model (305-1)) is upgradable via wireless firmware updates.

According to one embodiment, the AI model (e.g., the second AI model (305-1) in FIG. 3) is trained not only on the user's biometric information but also on various types of information, such as user profile data (e.g., age, height, weight, gender, degree of developmental disorder), user movement information, user vision information, user location information, ambient noise information, ambient brightness information, weather information, or survey data completed by the user's guardian (e.g., the user's psychological condition), to determine the user's psychological state or stress level.

According to one embodiment, the external device (102) is referred to as a server that operates an artificial intelligence model (e.g., the second AI model (305-2) in FIG. 3) for determining an air pressure pattern to be applied to the user based on the user's biometric information. The external device (102) inputs the biometric information received in real time from the wearable device (101) into the AI model and determines the air pressure pattern to be applied to the user based on the output value generated by the model. The AI model (e.g., the second AI model (305-2)) is upgradable via wireless firmware updates.

According to one embodiment, the AI model (e.g., the second AI model (305-2) in FIG. 3) is trained not only on the user's biometric information but also on various types of information, such as user profile data (e.g., age, height, weight, gender, degree of developmental disorder), user movement information, user vision information, user location information, ambient noise information, ambient brightness information, weather information, air pressure pattern data, or user response information (e.g., changes in the user's biometric data after air pressure application), to determine in real time the optimal air pressure pattern to be provided to the user.

According to one embodiment, the electronic device (103) refers to a portable terminal (e.g., a smartphone). For example, the electronic device (103) may refer to a smartphone used by a guardian of the user wearing the wearable device (101). The electronic device (103) receives the biometric information of the user, who is wearing the wearable device (101), in real time from the wearable device (101). The electronic device (103) displays the received biometric information via a display included in the electronic device (103). The electronic device (103) also receives location information of the wearable device (101) in real time from the wearable device (101). The location information of the wearable device (101) may correspond substantially to the location of the user wearing the device. The electronic device (103) displays the location information of the wearable device (101) via the display.

According to one embodiment, the electronic device (103) receives various types of information from a plurality of wearable devices, including biometric information, location information, ambient noise information, ambient brightness information, vision information, or weather information. The electronic device (103) receives identification information from each of the plurality of wearable devices that enables the wearable devices to be individually identified, and based on the received identification information, the electronic device (103) separately receives various types of information for each wearable device, such as biometric information, location information, ambient noise, ambient brightness, vision, or weather conditions. For example, the electronic device (103) displays a first object on the display indicating the location information of a first wearable device among the plurality of wearable devices, and simultaneously displays a second object indicating the location information of a second wearable device on the same display.

According to one embodiment, the electronic device (103) simultaneously displays an object representing the location information of the wearable device (101) and various types of information associated with the object. For example, the electronic device (103) displays an object indicating the location of the wearable device (101) at a first position on the screen, and simultaneously displays, near the first position, information indicating the current psychological state or stress level associated with the object.

According to one embodiment, the electronic device (103) receives not only the user's biometric information but also various other types of information from the wearable device (101). The various types of information may include the user's location information, ambient noise information, ambient brightness information, or vision information of the user.

According to one embodiment, the electronic device (103) controls the wearable device (101). For example, based on a user input received via the electronic device (103) for controlling the air pump of the wearable device (101), the electronic device (103) injects air into or discharges air from the air tube.

According to one embodiment, the electronic device (103) controls the wearable device (101) such that the air tube included in the wearable device (101) applies pressure of a designated intensity to a designated region of the user's body, who is wearing the wearable device, for a designated period of time. The intensity and duration of the air pressure applied to the user may vary depending on the specific body region. For example, the wearable device (101) may apply pressure to the user's chest at a first pressure level for a first duration, and apply pressure to the user's shoulder at a second pressure level for a second duration. In one example, both the chest and the shoulder may simultaneously receive air pressure stimulation for respective designated durations and pressure levels. In another example, air pressure may first be applied to the chest at the first pressure level for the first duration, followed by air pressure applied to the shoulder at the second pressure level for the second duration. The electronic device (103) determines the order of body regions to which air pressure is applied.

According to one embodiment, the electronic device (103) displays a control history of the wearable device (101). For example, the electronic device (103) displays, in real time, control records including the activation date and time, intensity, duration, target body region, and the psychological state or stress level related to the operation of the air pressure in the wearable device (101).

According to one embodiment, the electronic device (103) includes an application installed thereon that operates a system for managing a user's psychological state based on biometric information. The application may be configured to execute various embodiments described in the present document.

FIG. 2 illustrates a block diagram of a wearable device (101) according to an embodiment of the present disclosure.

Referring to FIG. 2, the wearable device (101) includes a first processor (201), a first biometric sensor (203-1), a second biometric sensor (203-2), a first communication module (205-1), a second communication module (205-2), a third communication module (205-3), a biometric information processing module (206), an air pump (207), an air tube (209), a detachment detection sensor (211), an IMU sensor (213), and a camera module (215). However, the wearable device (101) is not limited to the components illustrated in FIG. 2, and some components may be omitted or additionally included. For example, the wearable device (101) may omit the detachment detection sensor (211), the IMU sensor (213), and the camera module (215) shown in FIG. 2. The first biometric sensor (203-1) may include the first communication module (205-1) and the biometric information processing module (206). The second biometric sensor (203-2) may include the second communication module (205-2). In another example, the wearable device (101) may further include a memory module, a battery module, and a pressure detection sensor in addition to the components illustrated in FIG. 2. The memory may store instructions for executing the methods according to embodiments of the present disclosure. The memory may include at least one of a volatile storage medium and a non-volatile storage medium. For example, the memory may include at least one of read-only memory (ROM) and random access memory (RAM). The pressure detection sensor may detect data related to the air pressure of the air tube (209) in the wearable device (101). For example, the wearable device (101) may detect an air pressure intensity, a change in the air pressure intensity, or an air pressure holding time of the air tube (209) through the pressure detection sensor. According to an embodiment, the wearable device (101) may further include a locking mechanism. The locking mechanism may be a buckle-type structure that prevents the user from voluntarily removing the wearable device (101) once it is worn. When the wearable device (101) is worn by the user, the locking mechanism may be in a locked state. If, while the locking mechanism of the wearable device (101) is in the locked state, the user's psychological state corresponds to a predetermined state, or the stress level is equal to or higher than a specified level, or the user's motion data value is greater than a threshold value, the wearable device (101) may determine that the user is experiencing discomfort while wearing the wearable device (101). In response, the wearable device (101) may change the state of the locking mechanism to an unlocked state.

According to an embodiment, the first processor (201) may refer to a processor in which the methods according to embodiments of the present disclosure are executed, such as a central processing unit (CPU), a graphics processing unit (GPU), or the like. The first processor (201) may be electrically and/or operably connected to the first biometric sensor (203-1), the second biometric sensor (203-2), the third communication module (205-3), the air pump (207), the detachment detection sensor (211), the IMU sensor (213), and the camera module (215). The first processor (201) may analyze and process various data acquired from the first biometric sensor (203-1), the second biometric sensor (203-2), the third communication module (205-3), the air pump (207), the detachment detection sensor (211), the IMU sensor (213), and the camera module (215), and may control the corresponding components based on the result of the analysis and processing.

According to an embodiment, the first biometric sensor (203-1) and the second biometric sensor (203-2) may be referred to as biometric sensors that acquire biometric information. The biometric information may include electrodermal activity (EDA), photoplethysmogram (PPG), blood volume pulse (BVP), respiration (RESP), thermal (or body temperature), heart rate variability (HRV), or heart rate (HR). The biometric information is not limited to the aforementioned examples and may include various types of biometric data used in determining the psychological state or stress level of a user.

According to an embodiment, the first biometric sensor (203-1) and the second biometric sensor (203-2) may each have dimensions of 40 mm in length, 20 mm in width, and 20 mm in height. The first biometric sensor (203-1) and the second biometric sensor (203-2) may acquire the user's biometric signals using radar signals having a wavelength of 60-64 GHz. When the first biometric sensor (203-1) and the second biometric sensor (203-2) are attached (or mounted) to the wearable device (101), they may sense biometric information within a range of 900 mm from the sensor module. When the first biometric sensor (203-1) and the second biometric sensor (203-2) are detached from the wearable device (101) and placed on a charging cradle, they may use a booster function to extend the sensing range to within 4000 mm.

According to an embodiment, the first biometric sensor (203-1) may include a first communication module (205-1) and a biometric information processing module (206). The first biometric sensor (203-1) may be referred to as a sensor module mounted on the front portion of the wearable device (101). The front portion may refer to the front side of a vest-type garment, assuming that the wearable device (101) is implemented as a garment capable of providing air pressure, and may correspond to the surface facing the chest of the user when the wearable device (101) is worn. The first communication module (205-1) may refer to a module that supports wireless communication between the first biometric sensor (203-1) and the external device (102) and the electronic device (103). The wireless communication may include LTE communication. The biometric information processing module (206) may refer to a module that processes biometric information acquired by the first biometric sensor (203-1) and the second biometric sensor (203-2), respectively. The first biometric sensor (203-1) may be understood as a module that further includes the biometric information processing module (206), compared to the configuration of the second biometric sensor (203-2), and the first biometric sensor (203-1) and the second biometric sensor (203-2) may be respectively understood as a master device and a slave device.

According to an embodiment, the second biometric sensor (203-2) may include a second communication module (205-2). The second biometric sensor (203-2) may be referred to as a sensor module mounted on the rear portion of the wearable device (101). The rear portion may refer to the back side of a vest-type garment, assuming that the wearable device (101) provides air pressure, and may correspond to the surface facing the back of the user when the wearable device (101) is worn. The second communication module (205-2) may refer to a module that supports wireless communication between the second biometric sensor (203-2) and the external device (102) and the electronic device (103). The wireless communication may include LTE communication.

According to an embodiment, the first biometric sensor (203-1) may acquire first information as biometric information of the user, and the second biometric sensor (203-2) may acquire second information as biometric information of the user. The first biometric sensor (203-1) and the second biometric sensor (203-2) may perform wireless communication with each other. The wireless communication may include LTE communication or Bluetooth communication. The second biometric sensor (203-2) may transmit the second information to the first biometric sensor (203-1) via wireless communication (e.g., Bluetooth communication). The first biometric sensor (203-1) may process the first information and the second information received from the second biometric sensor (203-2) through the biometric information processing module (206). For example, the first biometric sensor (203-1) may extract overlapping information between the first information and the second information through the biometric information processing module (206), and determine the extracted overlapping information as the biometric information of the user. By determining the biometric information of the user based on the information acquired from the two sensor modules, the accuracy of the biometric information of the user can be improved.

According to an embodiment, the third communication module (205-3) may support the wearable device (101) to perform wireless communication with the external device (102) and the electronic device (103). For example, the wearable device (101) may receive a control signal for controlling the air pump (207) from the external device (102) through the third communication module (205-3). The third communication module (205-3) may support LTE communication or BLE communication.

According to an embodiment, the air pump (207) may inject air into or remove air from the air tube (209) based on the control signal. The air tube (209) may be disposed on the front and rear portions of the wearable device (101), respectively. The air tube (209) may include a plurality of diaphragms so that air can be injected or removed for each specific region of the front or rear portion of the wearable device (101). Accordingly, only the upper or lower portion of the front or rear side of the wearable device (101) may receive air injection.

According to an embodiment, the detachment detection sensor (211) may detect whether a sensor module (e.g., the first biometric sensor (203-1) or the second biometric sensor (203-2)) is detached from or attached to the wearable device (101).

According to an embodiment, the IMU sensor (213) may detect motion information or posture information of the wearable device (101). As the wearable device (101) moves in accordance with the movement of the user wearing the device, the IMU sensor (213) may measure motion information of the wearable device (101). The motion information or posture information of the wearable device (101) may be substantially identical to the motion or posture information of the user wearing the device and may be used interchangeably.

According to an embodiment, the camera module (215) may be provided on the front portion of the wearable device (101) to capture an environment corresponding to the user's line of sight when the wearable device (101) is worn by the user. The field of view (FOV) of the camera module (215) may be aligned with the user's line of sight and may be configured accordingly on the front portion of the wearable device (101). The wearable device (101) may acquire field-of-view information of the user by using the camera module (215).

FIG. 3 illustrates a block diagram of an external device (102) according to an embodiment.

Referring to FIG. 3, the external device (102) may include a second processor (301), a memory (303), an artificial intelligence (AI) processing unit (305), and a fourth communication module (307). The memory (303) may include a first memory (303-1), a second memory (303-2), and a third memory (303-3). The AI processing unit (305) may include a first AI model (305-1) and a second AI model (305-2). However, the external device (102) is not limited to the components illustrated in FIG. 3, and certain components may be omitted or additionally included. For example, the external device (102) may further include an AI model training unit in addition to the components illustrated in FIG. 3. The AI model training unit may be configured to train an AI model using various training data. The first AI model (305-1) and the second AI model (305-2) may be AI models trained by the AI model training unit.

According to an embodiment, the second processor (301) may be a processor in which methods according to embodiments of the present disclosure are executed, and may include a central processing unit (CPU), a graphics processing unit (GPU), or the like. The second processor (301) may be electrically and/or operably connected to the memory (303), the AI processing unit (305), and the fourth communication module (307). The second processor (301) may analyze and process various data obtained from the memory (303), the AI processing unit (305), and the fourth communication module (307), and may control the corresponding components based on the analysis and processing results.

According to an embodiment, the first memory (303-1) may store user information or personal information about a user wearing the wearable device (101), such as age, height, weight, gender, or the degree of developmental disorder. The external device (102) may receive and store the user information or personal information from the electronic device (103).

According to an embodiment, the second memory (303-2) may store biometric information (e.g., electrodermal activity (EDA), photoplethysmogram (PPG), blood volume pulse (BVP), respiration rate (RESP), thermal (or body temperature), heart rate variability (HRV), or heart rate (HR)), motion information, current time information, user location information, brightness information around the user, and noise information around the user, with respect to the user wearing the wearable device (101).

According to an embodiment, the third memory (303-3) may store information on the psychological state or stress level of the user wearing the wearable device (101). The information on the psychological state or stress level may be determined by the first artificial intelligence model (305-1) of the external device (102).

According to an embodiment, the AI processing unit (305) may include the first AI model (305-1) and the second AI model (305-2). The external device (102) may operate the first AI model (305-1) and the second AI model (305-2) based on various information received from the wearable device (101) and the electronic device (103), and may determine a psychological state or a stress level of the user wearing the wearable device (101), and an optimal air pressure pattern to stabilize the psychological state of the user. The term “stabilize the psychological state” may refer to a state in which the values included in the biometric information acquired in real time fall within a numerical range corresponding to a stable psychological state.

According to an embodiment, the AI processing unit (305) may train the first AI model (305-1) and the second AI model (305-2) by inputting various training data using an unsupervised anomaly detection technique. The first AI model (305-1) and the second AI model (305-2) may be operated using a convolutional neural network (CNN) and a support vector machine (SVM).

According to an embodiment, the first AI model (305-1) may be a model trained to determine a psychological state or a stress level of a user by using at least one of biometric information of the user (e.g., electrodermal activity (EDA), photoplethysmogram (PPG), blood volume pulse (BVP), respiration rate (RESP), thermal (or body temperature), heart rate variability (HRV), or heart rate (HR)), user information (e.g., age, height, weight, gender, degree of developmental disorder), user motion information, user view information, user location information, ambient noise information, ambient brightness information, weather information, or questionnaire data prepared by a caregiver of the user as training data. The questionnaire data prepared by the caregiver of the user may be data input through the electronic device (103) by the caregiver when it is determined that the user is experiencing psychological anxiety (e.g., when numerical values included in the biometric information deviate from the normal range), and may include data related to the user's psychological state or stress level, air pressure intensity, time, or area to be pressurized.

According to an embodiment, the first AI model (305-1) may be configured to output a psychological state or a stress level of the user as output data based on a value output by using at least one of biometric information, user information, motion information, view information, location information, ambient noise information, ambient brightness information, or weather information as input data.

According to an embodiment, the second artificial intelligence (AI) model (305-2) may be an AI model trained to determine an optimal air pressure pattern to be provided to the user, by using at least one of biometric information of the user (e.g., electrodermal activity (EDA), photoplethysmogram (PPG), blood volume pulse (BVP), respiration rate (RESP), thermal (or body temperature), heart rate variability (HRV), or heart rate (HR)), a psychological state or stress level of the user determined by the first AI model (305-1), user information (e.g., age, height, weight, gender, and degree of developmental disorder), motion information of the user, view information of the user, location information of the user, ambient noise information, ambient brightness information, weather information, air pressure pattern information, or user response information (e.g., biometric information of the user changed after air pressure is applied) as training data. The factors for determining the air pressure pattern may include air pressure intensity, air pressure duration, a body part on which air pressure is applied, and a sequence of body parts on which air pressure is to be applied.

According to an embodiment, the second AI model (305-2) may be configured to output data on an optimal air pressure pattern for the user, based on an output value obtained by using at least one of biometric information, a psychological state or stress level of the user determined by the first AI model (305-1), user information, motion information, view information, location information, ambient noise information, ambient brightness information, or weather information as input data. The optimal air pressure pattern data may refer to data about a pattern that enables the user's psychological state or stress level to effectively return to a normal range. For example, if a value included in the user's biometric information changes to fall within the normal range within a designated time after air pressure is applied in a specific pattern, such a pattern may be regarded as optimal. The shorter the time it takes for the value to return to the normal range, the more efficient the air pressure pattern may be considered.

According to an embodiment, the fourth communication module (307) may support the external device (102) in performing wireless communication with the wearable device (101) and the electronic device (103). The external device (102) may transmit and receive various types of information stored in the memory to and from the wearable device (101) and the electronic device (103) via the fourth communication module (307). The external device (102) may transmit and receive, to and from the wearable device (101) and the electronic device (103), information on the user's psychological state or stress level and information on an optimal air pressure pattern as determined by the first AI model (305-1) and the second AI model (305-2). Based on the information on the user's psychological state or stress level and the information on the optimal air pressure pattern as determined by the first AI model (305-1) and the second AI model (305-2), the external device (102) may transmit and receive control information for controlling the air pump (207) of the wearable device (101) via the fourth communication module (307).

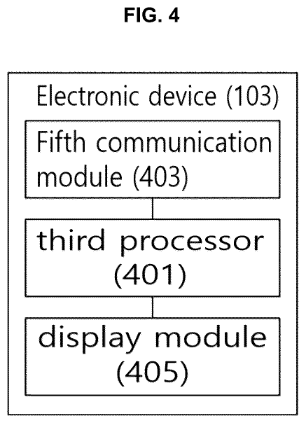

FIG. 4 illustrates a block diagram of an electronic device (103) according to an embodiment.

Referring to FIG. 4, the electronic device (103) may include a third processor (401), a fifth communication module (403), and a display module (405). However, the electronic device (103) is not limited to the components illustrated in FIG. 4, and some components may be omitted or additionally included. For example, the electronic device (103) may further include a microphone and a speaker module. When the number of times air pressure is applied to the user wearing the wearable device (101) exceeds a designated number, the electronic device (103) may output a warning notification through the speaker module.

According to an embodiment, the third processor (401) may refer to a processor that performs methods according to embodiments of the present disclosure, such as a central processing unit (CPU) or a graphics processing unit (GPU). The third processor (401) may be electrically and/or operably connected to the fifth communication module (403) and the display module (405). The third processor (401) may analyze and process various types of data acquired from the fifth communication module (403) and the display module (405), and may control the corresponding components based on the result of the analysis and processing.

According to an embodiment, the fifth communication module (403) may support the electronic device (103) in performing wireless communication with the wearable device (101) and the external device (102).

According to an embodiment, the display module (405) may display various types of information. For example, the electronic device (103) may display biometric information received from the wearable device (101) through the display module (405).

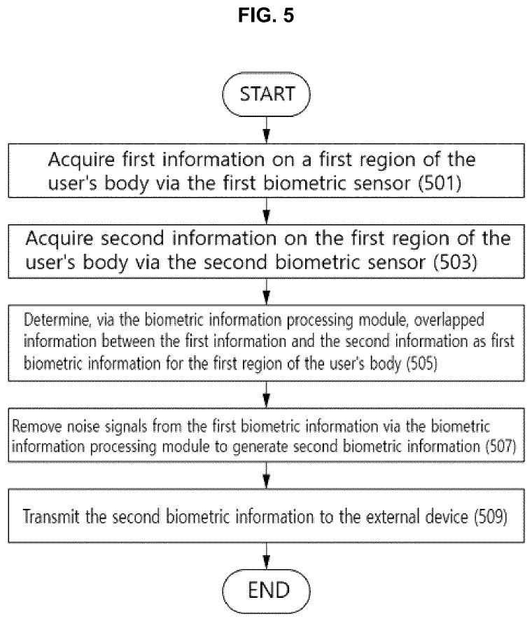

FIG. 5 illustrates a flowchart of a method for acquiring biometric information of a user by a wearable device (101) according to an embodiment.

The operations of the wearable device (101) described below may be performed in a different order or simultaneously.

In operation 501, the wearable device (101) acquires first information regarding a first region of a user's body via a first biometric sensor (203-1). The first biometric sensor (203-1) is a biometric sensor mounted on a front portion of the wearable device (101), and the first information may refer to biometric information of the user. For example, the wearable device (101) may acquire heart rate or heart rate variability data of the user's chest region via the first biometric sensor (203-1). The first biometric sensor (203-1) may transmit a radar signal toward the user's heart region from the front portion of the wearable device (101), and acquire the heart rate or heart rate variability data based on a signal reflected from the heart region. The heart rate or heart rate variability data may include waveform data in the form of a sine function. In another example, the wearable device (101) may acquire respiration rate data of the user's lung region via the first biometric sensor (203-1). The first biometric sensor (203-1) may transmit a radar signal toward the lung region from the front portion of the wearable device (101), and acquire the respiration rate data based on a signal reflected from the lung region. The respiration rate data may include waveform data in the form of a sine function.

In operation 503, the wearable device (101) acquires second information regarding the first region of the user's body via a second biometric sensor (203-2). The second biometric sensor (203-2) is a biometric sensor mounted on a rear portion of the wearable device (101), and the second information may refer to biometric information of the user. For example, the wearable device (101) may acquire heart rate or heart rate variability data of the heart region via the second biometric sensor (203-2). The second biometric sensor (203-2) may transmit a radar signal toward the heart region from the rear portion of the wearable device (101), and acquire the heart rate or heart rate variability data based on a signal reflected from the heart region. The heart rate variability data may include waveform data in the form of a sine function. In another example, the wearable device (101) may acquire respiration rate data of the user's lung region via the second biometric sensor (203-2). The second biometric sensor (203-2) may transmit a radar signal toward the lung region from the rear portion of the wearable device (101), and acquire the respiration rate data based on a signal reflected from the lung region. The respiration rate data may include waveform data in the form of a sine function.

In operation 505, the wearable device (101) determines overlapping information between the first information and the second information as first biometric information regarding the first region of the user's body via a biometric information processing module (206) of the first biometric sensor (203-1). For example, the biometric information processing module (206) of the first biometric sensor (203-1) may determine the overlapped data between the heart rate or heart rate variability data (e.g., first information) acquired from the first biometric sensor (203-1) on the front portion of the wearable device (101) and the heart rate or heart rate variability data (e.g., second information) acquired from the second biometric sensor (203-2) on the rear portion of the wearable device (101), as biometric data (e.g., first biometric information) representing the user's heart region.

In operation 507, the wearable device (101) removes noise signals from the first biometric information to generate second biometric information via the biometric information processing module (206). The wearable device (101) may use a noise reduction filter, such as a Kalman filter, through the biometric information processing module (206) to remove the noise signals from the first biometric information.

In operation 509, the wearable device (101) transmits the second biometric information to an external device (102) via a third communication module (205-3). The external device (102) may store the second biometric information received from the wearable device (101) in a second memory (303-2).

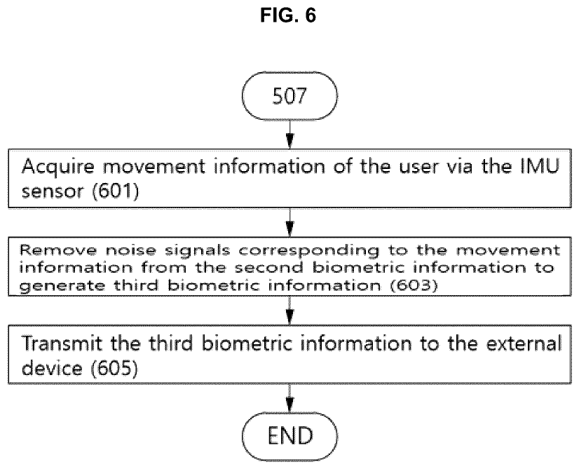

FIG. 6 illustrates a flowchart of a method for removing noise from biometric information based on motion information of a user by a wearable device (101) according to an embodiment.

The operations of the wearable device (101) described below may be performed in a different order or simultaneously.

In operation 601, the wearable device (101) acquires motion information of the user by using an IMU sensor (213) (or an acceleration sensor). The motion information may include movement direction, movement speed, posture information, vibration information indicating the degree of tremor in a body part of the user, and rotation information indicating the direction and speed of rotation of a body part of the user. The wearable device (101) may determine an average value of the motion data by dividing the total amount of motion data acquired during a specified time interval by the time interval value.

In operation 603, the wearable device (101) removes noise signals corresponding to the motion information from the second biometric information to generate third biometric information. For example, the wearable device (101) may remove noise signals corresponding to the average value of the motion data to generate the third biometric information.

In operation 605, the wearable device (101) transmits the third biometric information to an external device (102) via a third communication module (205-3). The external device (102) may store the third biometric information received from the wearable device (101) in a second memory (303-2) via a fourth communication module (307).

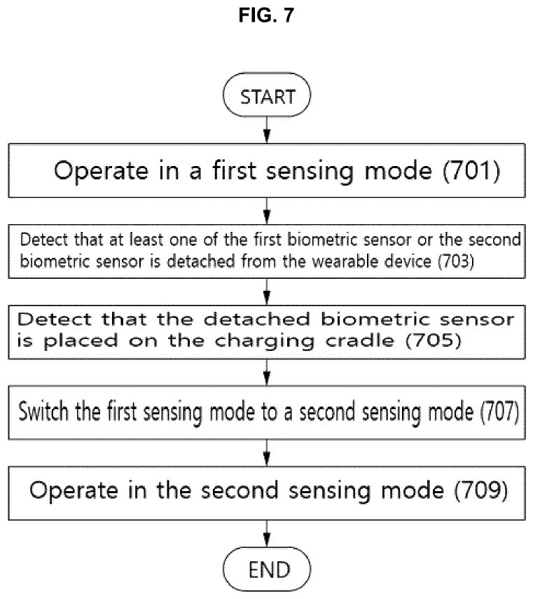

FIG. 7 illustrates a flowchart of a method for switching a sensing mode based on detachment of a biometric sensor from a wearable device (101) according to an embodiment.

The operations of the wearable device (101) described below may be performed in a different order or simultaneously.

In operation 701, the first biometric sensor (203-1) and the second biometric sensor (203-2) of the wearable device (101) may operate in a first sensing mode. The first sensing mode may refer to a state in which a biometric sensor (e.g., the first biometric sensor (203-1) or the second biometric sensor (203-2)) is attached to the wearable device (101) and operates to acquire biometric information within a first designated range (e.g., 900 mm).

In operation 703, the wearable device (101) may detect, through the detachment detection sensor (211), that at least one of the first biometric sensor (203-1) or the second biometric sensor (203-2) has been detached from the wearable device (101).

In operation 705, the charging cradle may detect that the detached biometric sensor has been placed in the charging cradle. The operation of detecting that the detached biometric sensor has been placed in the charging cradle may include detecting identification information of the biometric sensor placed in the cradle. The charging cradle may transmit a signal including information indicating that the detached biometric sensor has been placed in the cradle to the biometric sensor, based on the identification information.

In operation 707, the biometric sensor that receives the signal may switch from the first sensing mode to a second sensing mode. The second sensing mode may refer to a state in which a biometric sensor (e.g., the first biometric sensor (203-1) or the second biometric sensor (203-2)) is detached from the wearable device (101), placed in the charging cradle, and driven with a booster function activated to acquire biometric information within a second designated range (e.g., 4000 mm).

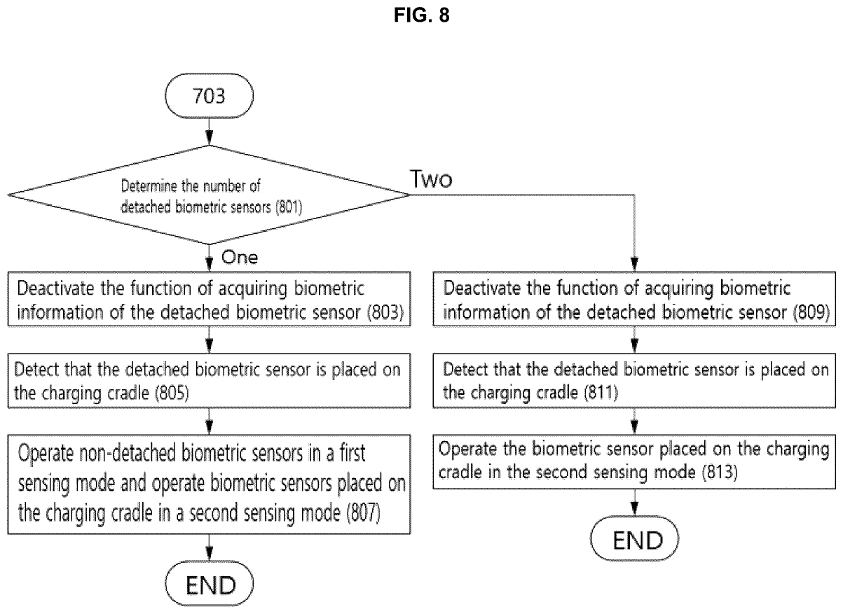

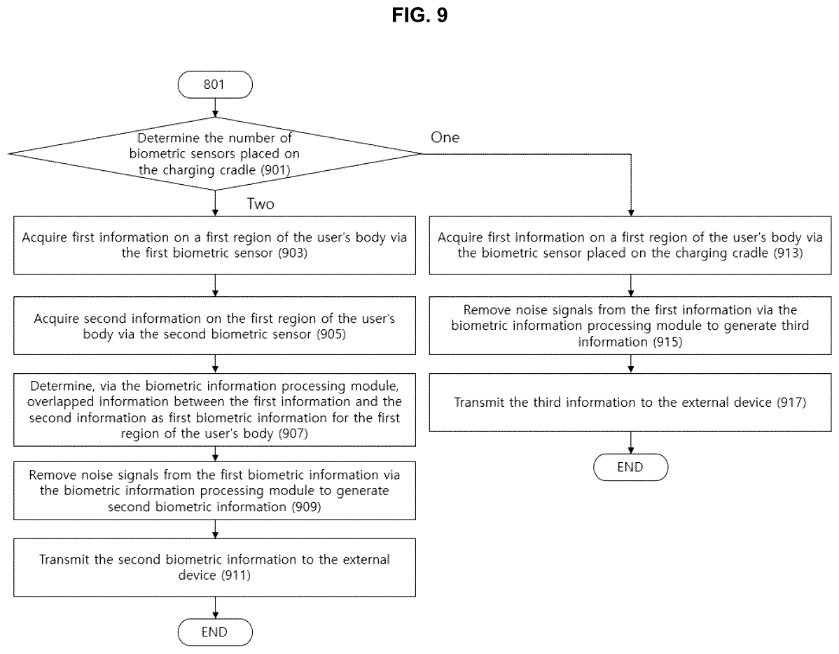

In operation 709, the biometric sensor that receives the signal may operate in the second sensing mode. Detailed descriptions of the method for operating the biometric sensor in the second sensing mode will be provided in FIGS. 8 and 9.

FIG. 8 illustrates a flowchart of a method in which a biometric sensor senses biometric information based on the number of biometric sensors detached from the wearable device (101), according to an embodiment.

The operations of the wearable device (101) described below may be performed in a different order or simultaneously.

In operation 801, after detecting, through the detachment detection sensor (211), that at least one of the first biometric sensor (203-1) or the second biometric sensor (203-2) has been detached from the wearable device (101), the wearable device (101) may identify the number of detached biometric sensors. According to an embodiment, if the number of detached biometric sensors is one, the detached biometric sensor may perform operation 803. If the number of detached biometric sensors is two, the detached biometric sensors may perform operation 809.

In operation 803, if the number of detached biometric sensors is one, the wearable device (101) may transmit a first signal to the detached biometric sensor to deactivate the function of acquiring biometric information. The detached biometric sensor may deactivate the function of acquiring biometric information in response to receiving the first signal.

In operation 805, the charging cradle may detect that the detached biometric sensor has been placed in the cradle. In response to detecting that the biometric sensor has been placed in the cradle, the charging cradle may transmit a second signal to the biometric sensor placed in the cradle to activate the function of acquiring biometric information. The biometric sensor that receives the second signal may activate the function of acquiring biometric information.

In operation 807, the biometric sensor that has not been detached from the wearable device (101) may operate in the first sensing mode. The biometric sensor placed in the charging cradle and having received the second signal may operate in the second sensing mode. In response to detecting that the biometric sensor has been placed in the charging cradle, the cradle may recognize a pre-registered user's face through a camera included in the cradle. For example, if multiple people are present in the area where the cradle is installed, the camera of the cradle may recognize the pre-registered user's face among the crowd, and transmit user-related information (e.g., user location information) corresponding to the recognized face to the biometric sensor placed in the cradle. The biometric sensor, upon receiving the user-related information while placed in the cradle, may transmit radar signals toward the user based on the user's location information and thereby acquire biometric information of the user. The cradle may acquire biometric information corresponding to the recognized face of the user.

In operation 809, if the number of detached biometric sensors is two, the wearable device (101) may transmit a first signal to the detached biometric sensors (e.g., the first biometric sensor (203-1) and the second biometric sensor (203-2)) to deactivate the function of acquiring biometric information. The detached biometric sensors (e.g., the first biometric sensor (203-1) and the second biometric sensor (203-2)) may deactivate the function of acquiring biometric information in response to receiving the first signal.

In operation 811, the charging cradle may detect that the detached biometric sensors have been placed in the cradle. In response to detecting that the biometric sensors have been placed in the cradle, the charging cradle may transmit a second signal to the biometric sensors placed in the cradle to activate the function of acquiring biometric information. The biometric sensors that receive the second signal may activate the function of acquiring biometric information.

In operation 813, the biometric sensors placed in the cradle and having received the second signal may operate in the second sensing mode. In response to detecting that the biometric sensors have been placed in the cradle, the cradle may recognize a pre-registered user's face through a camera included in the cradle. For example, if multiple people are present in the area where the cradle is installed, the camera of the cradle may recognize the pre-registered user's face among the crowd, and transmit user-related information (e.g., user location information) corresponding to the recognized face to the biometric sensors placed in the cradle. The biometric sensors, upon receiving the user-related information while placed in the cradle, may transmit radar signals toward the user based on the user's location information and thereby acquire biometric information of the user. The cradle may acquire biometric information corresponding to the recognized face of the user.

FIG. 9 illustrates a flowchart of a method in which biometric sensors perform sensing based on the number of biometric sensors placed on a charging cradle after being detached from the wearable device (101), according to an embodiment.

The operations of the wearable device (101) described below may be performed in a different order or simultaneously.

In operation 901, after both biometric sensors (e.g., the first biometric sensor (203-1) and the second biometric sensor (203-2)) are detached from the wearable device (101), when two biometric sensors are placed on the charging cradle, the first biometric sensor (203-1) performs operation 903. When only one biometric sensor is placed on the charging cradle, the placed biometric sensor performs operation 913.

In operation 903, while the first biometric sensor (203-1) is placed on the charging cradle, the first biometric sensor (203-1) acquires first information on a first body part of the user recognized by the camera of the charging cradle in the second sensing mode.

In operation 905, while the second biometric sensor (203-2) is placed on the charging cradle, the second biometric sensor (203-2) acquires second information on the first body part of the user recognized by the camera of the charging cradle in the second sensing mode.

In operation 907, the second biometric sensor (203-2) transmits the second information to the first biometric sensor (203-1). For example, the second biometric sensor (203-2) may transmit the second information to the first biometric sensor (203-1) via Bluetooth communication. The first biometric sensor (203-1) determines overlapped information between the first information and the second information using the biometric information processing module (206). The first biometric sensor (203-1) determines the overlapped information as first biometric information on the first body part of the user using the biometric information processing module (206).

In operation 909, the first biometric sensor (203-1) removes noise signals from the first biometric information to generate second biometric information using the biometric information processing module (206). For example, the first biometric sensor (203-1) removes noise signals included in the first biometric information using a Kalman filter through the biometric information processing module (206).

In operation 911, the first biometric sensor (203-1) transmits the second biometric information to the external device (102) via the first communication module (205-1).

In operation 913, the biometric sensor placed on the charging cradle acquires first information on the first body part of the user recognized by the camera of the charging cradle in the second sensing mode.

In operation 915, when the biometric sensor placed on the charging cradle is the first biometric sensor (203-1), the first biometric sensor (203-1) removes noise signals from the first information to generate third information using the biometric information processing module (206). For example, the first biometric sensor (203-1) removes noise signals included in the first biometric information using a Kalman filter through the biometric information processing module (206).

In operation 917, when the biometric sensor placed on the charging cradle is the first biometric sensor (203-1), the first biometric sensor (203-1) transmits the third information to the external device (102). When the biometric sensor placed on the charging cradle is the second biometric sensor (203-2), the second biometric sensor (203-2) transmits the first information to the external device (102) without performing the noise removal operation.

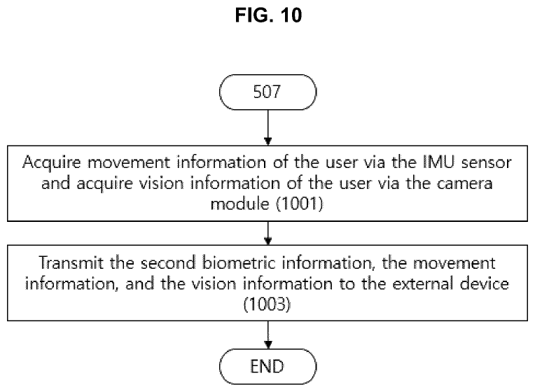

FIG. 10 illustrates a flowchart of a method in which the wearable device (101) transmits training data and input data for an artificial intelligence (AI) model to an external device (102) that executes the AI model for determining a psychological state, according to an embodiment.

The operations of the wearable device (101) described below may be performed in a different order or simultaneously.

In operation 1001, the wearable device (101) acquires motion information of the user through the IMU sensor (213), and acquires the user's field-of-view (FOV) information through the camera module (215). The motion information may include movement direction of the user, movement speed, posture information of the user, vibration information indicating the degree of trembling of a body part of the user, and rotation information indicating the rotation direction and speed of a body part of the user. The field-of-view information may include the direction of the user's gaze, the direction in which the gaze shifts, and the speed toward the shifted direction.

In operation 1003, the wearable device (101) transmits the second biometric information, the motion information, and the field-of-view information to the external device (102). The external device (102) may train or operate the first AI model (305-1) and the second AI model (305-2) using the second biometric information, the motion information, and the field-of-view information.

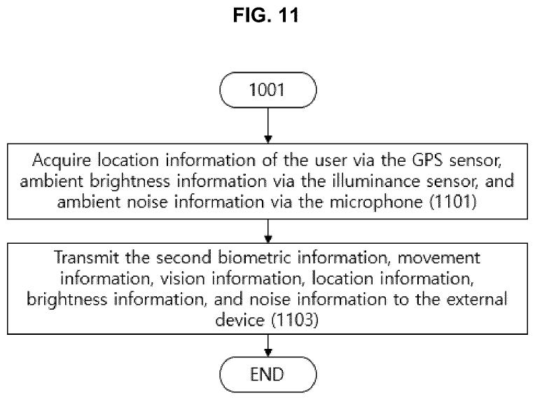

FIG. 11 illustrates a flowchart of a method in which a wearable device (101) transmits training data and input data for an artificial intelligence (AI) model to an external device (102) that executes the AI model for determining a psychological state, according to an embodiment.

The operations of the wearable device (101) described below may be performed in a different order or simultaneously.

In operation 1101, the wearable device (101) acquires location information of the user through a GPS sensor, acquires ambient brightness information through an illuminance sensor, and acquires ambient noise information through a microphone. While the user wearing the wearable device (101) moves outdoors, the wearable device (101) may acquire the location information of the user by utilizing Standalone GPS (S-GPS) through the GPS sensor. While the user wearing the wearable device (101) moves indoors, the wearable device (101) may acquire the location information of the user by utilizing Assisted GPS (A-GPS) of LTE through the GPS sensor.

In operation 1103, the wearable device (101) transmits the second biometric information, the motion information, the field-of-view information, the location information, the ambient brightness information, and the ambient noise information to the external device (102). The external device (102) may train or operate the first AI model (305-1) and the second AI model (305-2) using the second biometric information, the motion information, the field-of-view information, the location information, the ambient brightness information, and the ambient noise information.

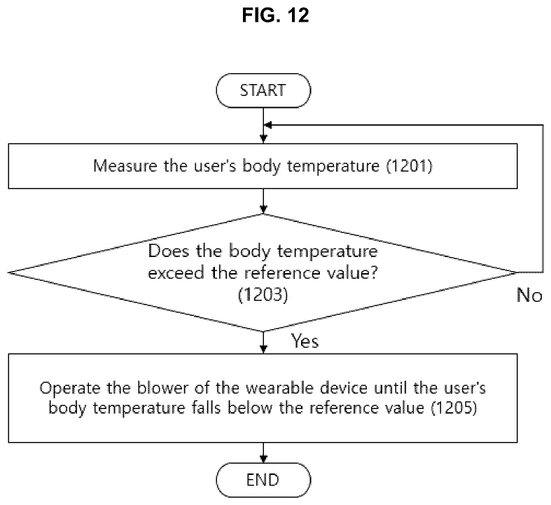

FIG. 12 illustrates a flowchart of a method for driving a blowing device of a wearable device (101) according to an embodiment.

The operations of the wearable device (101) described below may be performed in a different order or simultaneously.

In operation 1201, the wearable device (101) may include a temperature sensor. The wearable device (101) may measure a body temperature of a user wearing the wearable device (101) using the temperature sensor.

In operation 1203, the wearable device (101) may determine whether the body temperature exceeds a reference value. If the body temperature does not exceed the reference value, the wearable device (101) may perform operation 1201. If the body temperature exceeds the reference value, the wearable device (101) may perform operation 1205.

In operation 1205, the wearable device (101) may drive the blowing device of the wearable device (101) until the body temperature of the user becomes lower than the reference value. The wearable device (101) may include a plurality of through-holes respectively formed in a front portion and a rear portion. The front portion and the rear portion of the wearable device (101) may be formed of a mesh material. By being configured with a mesh material, having through-holes, and driving the blowing device, the wearable device (101) may regulate the user's body temperature.

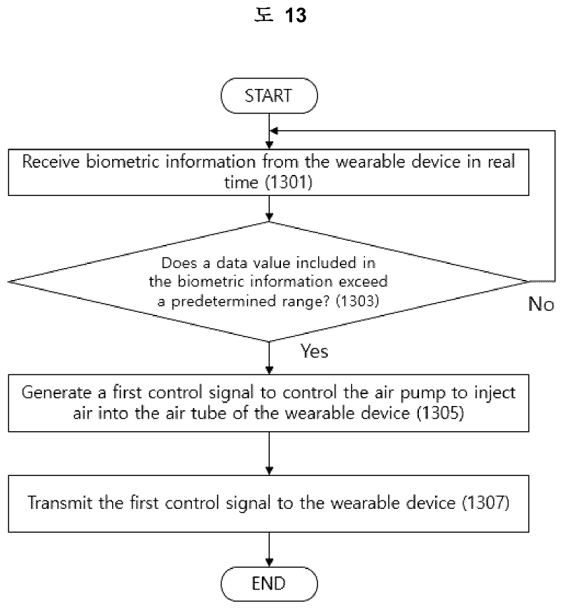

FIG. 13 illustrates a flowchart of a method for transmitting a first control signal from an external device (102) to a wearable device (101) to control an air pump (207) to inject air into an air tube (209) of the wearable device (101), according to an embodiment.

The operations of the external device (102) described below may be performed in a different order or simultaneously.

In operation 1301, the external device (102) may receive biometric information from the wearable device (101) in real time. The biometric information may be information acquired through a biosensor (e.g., the first biosensor (203-1) or the second biosensor (203-2)) of the wearable device (101).

In operation 1303, the external device (102) may determine whether a data value included in the biometric information exceeds a specified range. For example, the biometric information may include heart rate (HR), and the specified range may be between a first value and a second value, representing a normal heart rate range by age. For instance, the normal average heart rate for adults aged 20 or older is approximately 70 to 75 beats per minute, so the first value may be 70 and the second value may be 75. However, the numerical values are not limited to these examples. As another example, the biometric information may include heart rate variability (HRV), and the specified range may represent a normal HRV range by age. For instance, when the psychological state is stable or the stress level is low, HRV may be high, and in this case, the difference between the first and second values in the specified range may correspond to a first difference value. When the psychological state is unstable or the stress level is high, HRV may be low, and the difference in that case may be a second difference value. The first difference value may be greater than the second difference value. As yet another example, the biometric information may include respiratory rate (RESP), and in this case, the first value may be 12 breaths per minute and the second value may be 20 breaths per minute. As another example, the biometric information may include body temperature, and in this case, the first value may be 36° C. and the second value may be 37.5° C.

In operation 1305, when it is determined that the data value included in the biometric information exceeds the specified range, the external device (102) may generate a first control signal to control the air pump (207) to inject air into the air tube (209) of the wearable device (101). The first control signal may include information such as air pressure intensity, air pressure duration, and the body part to be pressurized. For example, the first control signal may set the air pressure intensity in the air tube (209) to a first intensity and set the duration from air injection to air release to a first duration, thereby controlling the air pump (207) to inject air into the part of the air tube (209) corresponding to the user's chest to apply pressure to the chest.

In operation 1307, the external device (102) may transmit the first control signal to the wearable device (101) via the fourth communication module (307). The wearable device (101) may control the air pump (207) to inject air into the air tube (209) based on the first control signal.

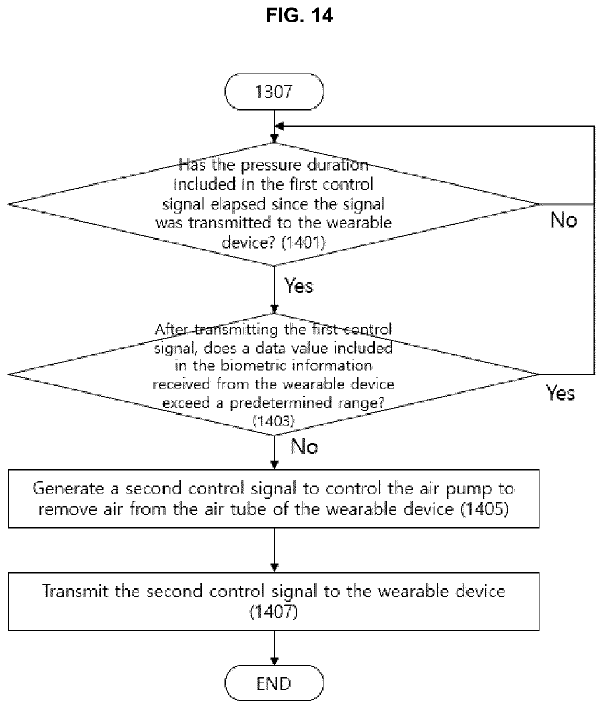

FIG. 14 illustrates a flowchart of a method for transmitting a second control signal from an external device (102) to a wearable device (101) to control an air pump (207) to remove air from an air tube (209) of the wearable device (101), according to an embodiment.

The operations of the external device (102) described below may be performed in a different order or simultaneously.

In operation 1401, the external device (102) may determine whether an air compression duration included in a first control signal has elapsed since the transmission of the first control signal to the wearable device (101) to control the air pump (207) to inject air into the air tube (209). For example, if the first control signal includes a command to control the air pump (207) to inject air into the air tube (209) for a first duration, the first duration may correspond to the air compression duration.

In operation 1403, after the air compression duration has elapsed since the first control signal was transmitted to the wearable device (101), the external device (102) may determine whether a data value included in biometric information collected after that time exceeds a specified range. Except for the point that the biometric information was collected after the air compression duration has elapsed since the transmission of the first control signal, operation 1403 may be substantially the same as operation 1303.

In operation 1405, when the external device (102) determines that the data value included in the biometric information collected after the air compression duration has elapsed does not exceed the specified range, the external device (102) may generate a second control signal to control the air pump (207) to remove air from the air tube (209) of the wearable device (101).

In operation 1407, the external device (102) may transmit the second control signal to the wearable device (101) via the fourth communication module (307). The wearable device (101) may control the air pump (207) to remove air from the air tube (209) based on the second control signal received from the external device (102).

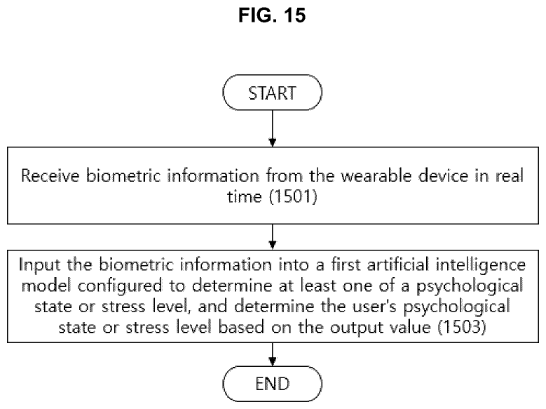

FIG. 15 illustrates a flowchart of a method for determining a user's psychological state using a first artificial intelligence (AI) model (305-1) by an external device (102), according to an embodiment.

The operations of the external device (102) described below may be performed in a different order or simultaneously.

In operation 1501, the external device (102) may receive biometric information from the wearable device (101) in real time.

In operation 1503, the external device (102) may input the biometric information into the first AI model (305-1), which is configured to determine at least one of a psychological state or a stress level, and may determine the user's psychological state or stress level based on an output value. The psychological state may include emotions such as anger, sadness, joy, and calmness. The stress level may be categorized into a plurality of stages. For example, the stress level may be classified from a first level to a fifth level, where the degree of stress may increase from the first to the fifth level. The first level may be interpreted as a stage with no stress, and from the second level onward, it may be considered a stage where stress exists and management is needed.

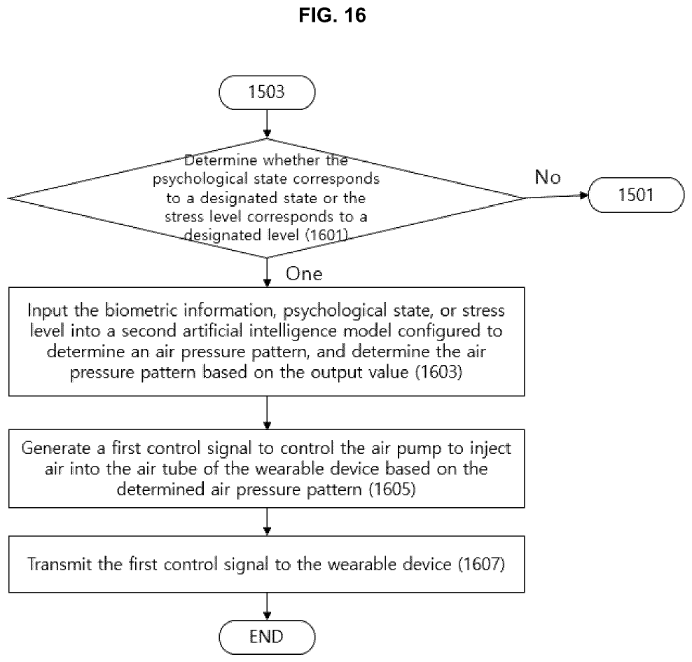

FIG. 16 illustrates a flowchart of a method for transmitting a first control signal from an external device (102) to a wearable device (101) to control an air pump (207) to inject air into an air tube (209) based on a pattern of air compression determined using a first artificial intelligence (AI) model (305-1) and a second AI model (305-2), according to an embodiment.

The operations of the external device (102) described below may be performed in a different order or simultaneously.

In operation 1601, the external device (102) may determine whether the user's psychological state corresponds to a designated psychological state or whether the user's stress level corresponds to a designated level using the first AI model (305-1).

In operation 1603, when the user's psychological state determined using the first AI model (305-1) corresponds to a first designated state (e.g., anger, sadness), or when the user's stress level (e.g., from level 1 to level 5) corresponds to a designated level (e.g., level 2 to level 5), the external device (102) may determine that deep pressure stimulation should be provided to the user by injecting air into the air tube (209) of the wearable device (101) using the air pump (207).

According to an embodiment, if the user's psychological state or stress level corresponds to the designated condition, the external device (102) may input at least one of the biometric information, the psychological state, or the stress level into the second AI model (305-2) and determine a pattern of air compression based on the output.

According to an embodiment, the first AI model (305-1) may be trained using at least one of user biometric information (e.g., electrodermal activity (EDA), photoplethysmography (PPG), blood volume pulse (BVP), respiration rate (RESP), thermal data (or body temperature), heart rate variability (HRV), or heart rate (HR)), user profile information (e.g., age, height, weight, gender, developmental disorder level), user movement information, user gaze information, user location information, ambient noise information, ambient light level, weather data, or caregiver-provided survey data (e.g., psychological state, stress level). The caregiver-provided data may include input via the electronic device (103) when the caregiver determines that the user is experiencing psychological discomfort, such as when biometric values are outside a normal range, and may include information regarding psychological state, stress level, air pressure intensity, duration, or targeted body area.

In another embodiment, the first AI model (305-1) may be configured to output a psychological state or stress level based on at least one of the aforementioned inputs.

According to an embodiment, the second AI model (305-2) may be trained to determine an optimal air compression pattern to be applied to the user based on at least one of the user's biometric data, the psychological state or stress level determined by the first AI model (305-1), user profile information, user movement information, user gaze information, user location information, ambient noise information, ambient light level, weather data, previously applied air compression patterns, or user response information (e.g., changes in biometric data after compression). The compression pattern may include air pressure intensity, compression duration, targeted body region, and sequence of compression areas.

In another embodiment, the second AI model (305-2) may be configured to output an optimal air compression pattern based on at least one of the above types of input data.

In operation 1605, the external device (102) may generate a first control signal to control the air pump (207) to inject air into the air tube (209) of the wearable device (101), based on the air compression pattern determined by the second AI model (305-2).

In operation 1607, the external device (102) may transmit the first control signal to the wearable device (101) via the fourth communication module (307). The wearable device (101) may control the air pump (207) to inject air into the air tube (209) based on the received first control signal.

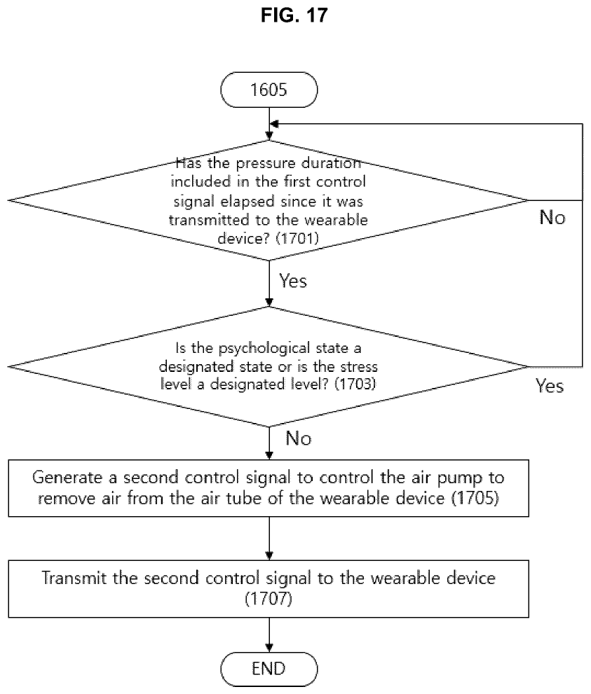

FIG. 17 illustrates a flowchart of a method for transmitting a second control signal from an external device (102) to a wearable device (101) to control an air pump (207) to release air from an air tube (209) based on the user's psychological state determined by a first artificial intelligence (AI) model (305-1), according to an embodiment.

The operations of the external device (102) described below may be performed in a different order or simultaneously.

In operation 1701, the external device (102) may determine whether a time duration specified as an air compression duration included in a first control signal has elapsed since the first control signal was transmitted to the wearable device (101) to control the air pump (207) to inject air into the air tube (209). For example, if the first control signal includes a command to operate the air pump (207) for a first duration to inject air into the air tube (209), the first duration may correspond to the air compression duration.

In operation 1703, after the air compression duration has elapsed from the time the first control signal was transmitted to the wearable device (101), the external device (102) may input collected data—such as biometric information, user profile information, movement information, gaze information, location information, ambient noise information, ambient brightness information, or weather data-into the first AI model (305-1), and determine whether the user's psychological state (e.g., anger, sadness, joy, relaxation) corresponds to a designated state (e.g., anger, sadness), or whether the user's stress level (e.g., from level 1 to level 5) corresponds to a designated level (e.g., level 2 to level 5), based on the output of the model.

In operation 1705, if the psychological state determined based on the output of the first AI model (305-1) does not correspond to the designated state (e.g., anger, sadness), and the stress level does not correspond to the designated level (e.g., level 2 to level 5), the external device (102) may generate a second control signal to control the air pump (207) to release air from the air tube (209) of the wearable device (101).

In operation 1707, the external device (102) may transmit the second control signal to the wearable device (101) via the fourth communication module (307). The wearable device (101) may control the air pump (207) to release air from the air tube (209) based on the received second control signal.

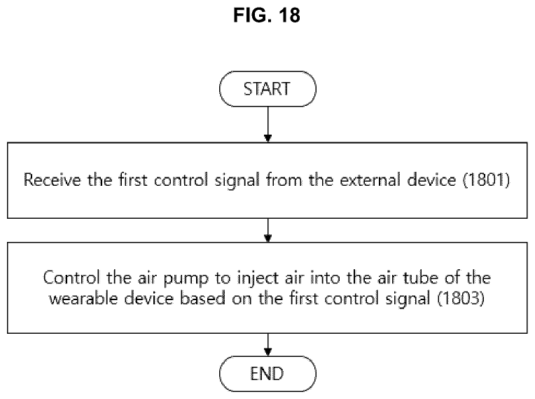

FIG. 18 illustrates a flowchart of a method for controlling an air pump (207) to inject air into an air tube (209) based on a first control signal by a wearable device (101), according to an embodiment.

The operations of the wearable device (101) described below may be performed in a different order or simultaneously.

In operation 1801, the wearable device (101) may receive a first control signal from an external device (102) via a third communication module (205-3). The first control signal may include a command to inject air into the air tube (209) of the wearable device (101). The first control signal may include information regarding compression intensity, compression duration, compression region, and the order of compression regions. For example, the first control signal may include a command to compress a first body part of the user (e.g., the chest) with a first compression intensity for a first duration and then compress a second body part of the user (e.g., the shoulder) with a second compression intensity for a second duration. In another example, the first control signal may instruct to compress the same first body part (e.g., the chest) sequentially with a first compression intensity for a first duration, followed by a second compression intensity for a second duration. The combinations of compression intensity, duration, body region, and order are not limited to the aforementioned examples. For instance, the same body part (e.g., the chest) may be compressed at varying intensities at specific time intervals, or the intensity may increase or decrease gradually, or alternate in intensity at predefined intervals. Additionally, different air compression patterns may be applied based on stress levels. For example, if the stress level corresponds to level 2, air compression may be applied with a first compression intensity for a first duration. If the stress level corresponds to level 3, a second compression intensity greater than the first may be applied for a second duration. That is, as the user's stress level increases, a higher air pressure may be applied to deliver deep pressure stimulation.

In operation 1803, the wearable device (101) may control the air pump (207) to inject air into the air tube (209) of the wearable device (101) based on the received first control signal.

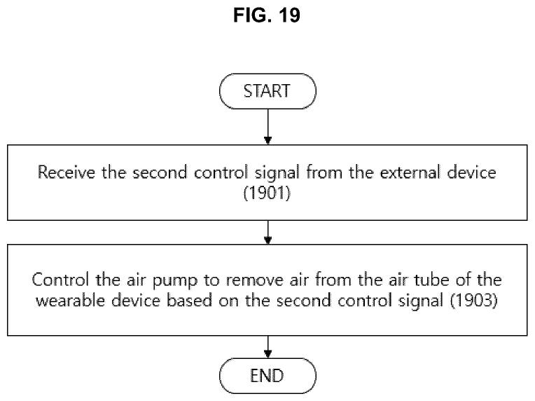

FIG. 19 illustrates a flowchart of a method for controlling an air pump (207) to remove air from an air tube (209) based on a second control signal by a wearable device (101), according to an embodiment.

The operations of the wearable device (101) described below may be performed in a different order or simultaneously.

In operation 1901, the wearable device (101) may receive a second control signal from an external device (102) via a third communication module (205-3). The second control signal may include a command to remove air from the air tube (209) of the wearable device (101).

In operation 1903, the wearable device (101) may control the air pump (207) to remove air from the air tube (209) of the wearable device (101) based on the received second control signal.

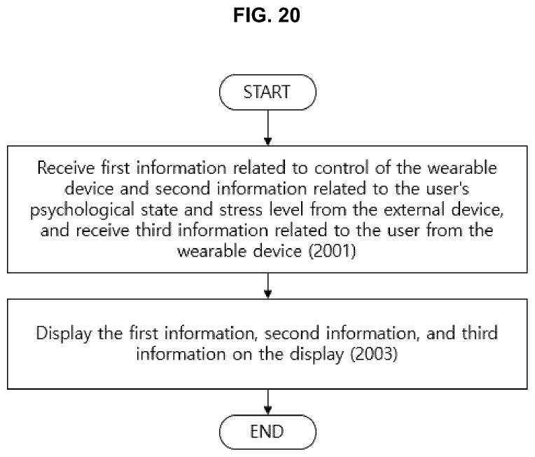

FIG. 20 illustrates a flowchart of a method for displaying various types of information by an electronic device (103), according to an embodiment.

The operations of the electronic device (103) described below may be performed in a different order or simultaneously.

In operation 2001, the electronic device (103) may receive first information related to control of the wearable device (101) (e.g., first control information or second control information), and second information related to the user's psychological state (e.g., anger, sadness, joy, relaxation) and stress level from an external device (102). The electronic device (103) may also receive third information related to the user from the wearable device (101). For example, the third information related to the user may include user information (e.g., age, height, weight, gender, degree of developmental disability) and biometric information of the user (e.g., electrodermal activity (EDA), photoplethysmogram (PPG), blood volume pulse (BVP), respiration rate (RESP), thermal data, heart rate variability (HRV), or heart rate (HR)).

In operation 2003, the electronic device (103) may display the first information, second information, and third information through a display module (405). The electronic device (103) may update and change the displayed content of the first, second, and third information in real time. In response to receiving a signal from the external device (102) indicating that the air pump (207) of the wearable device (101) was automatically controlled, the electronic device (103) may display the signal through the display module (405). According to an embodiment, the electronic device (103) may display identification information identifying the wearable device (101) in a first region located at an upper part of the screen via the display module (405). In a second region located below the first region, the electronic device (103) may display biometric information (e.g., heart rate, heart rate variability, body temperature, respiration rate), information on stress level, and information on psychological state. The electronic device (103) may display, from left to right in sequence, the biometric information, the stress level information, and the psychological state information in the second region. The electronic device (103) may display various average data in a third region located below the second region. For example, the various average data may include the average wearing time of the wearable device (101), the average air pressure intensity of the wearable device (101), and the user's average biometric information (e.g., average heart rate, average heart rate variability, average body temperature, average respiration rate).

What is claimed is:

1. A system for managing a psychological state of a user based on biometric information of the user, the system comprising:
a wearable device including an air tube configured to apply pressure to a portion of the user's body and an air pump configured to inject air into or discharge air from the air tube; and
an external device configured to operate an artificial intelligence model, the artificial intelligence model being configured to generate control information for controlling the air pump of the wearable device based on output data obtained by using the biometric information as input data, and being trained using the biometric information,
wherein the wearable device includes:
a first biometric sensor disposed at a front portion of the wearable device, configured to acquire the user's biometric information in a non-contact manner and including a biometric information processing module configured to process the biometric information;
a second biometric sensor disposed at a rear portion of the wearable device, configured to acquire the user's biometric information in a non-contact manner;
a first communication module operatively connected to the first biometric sensor;
a second communication module operatively connected to the second biometric sensor;
a third communication module; and
a processor electrically connected to the first biometric sensor, the second biometric sensor, the air pump, and the third communication module, wherein the processor is configured to:
acquire first information on a first region of the user's body via the first biometric sensor;
acquire second information on the same first region via the second biometric sensor;
determine overlapped information between the first information and the second information as first biometric information of the first region via the biometric information processing module;
remove noise signals from the first biometric information to generate second biometric information via the biometric information processing module; and
transmit the second biometric information to the external device via the first communication module.

2. The system of claim 1,
The wearable device further includes an inertial measurement unit (IMU) sensor, and
the processor is configured to:
acquire movement information of the user via the IMU sensor;
remove noise signals corresponding to the movement information from the second biometric information to generate third biometric information; and
transmit the third biometric information to the external device via the third communication module.

3. The system of claim 2,
the IMU sensor includes a 9-axis IMU sensor comprising a 3-axis accelerometer, a 3-axis gyroscope, and a 3-axis magnetometer.

4. The system of claim 1,
The system further includes a charging cradle comprising a wireless charging module configured to wirelessly charge a first battery of the first biometric sensor and a second battery of the second biometric sensor, and a camera,
wherein the wearable device further includes a detachment detection sensor, and
wherein the processor is configured to:
detect, via the detachment detection sensor, that at least one of the first biometric sensor or the second biometric sensor has been detached from the wearable device; and
in response to the detection that at least one of the first biometric sensor or the second biometric sensor has been detached, transmit, via the third communication module, a first signal to the detached biometric sensor to deactivate a function of acquiring biometric information by the detached biometric sensor.

5. The system of claim 4,
When the detached biometric sensor is placed on the charging cradle:
the detached biometric sensor activates the function of acquiring biometric information, and
wherein the charging cradle configured to:
recognize a user's face via the camera; and
provide, to the biometric sensor placed on the charging cradle, user-related information corresponding to the recognized face, and
wherein the biometric sensor placed on the charging cradle configured to:
acquire biometric information of the user corresponding to the recognized face based on the user-related information received from the charging cradle.

6. The system of claim 5,
When the biometric sensor placed on the charging cradle is the first biometric sensor:
wherein the first biometric sensor configured to:
acquire the first information while being placed on the charging cradle;
receive, via the first communication module, second information acquired by the second biometric sensor; and
determine, via the biometric information processing module, overlapped information between the first information and the second information as first biometric information for the first region of the user.

7. The system of claim 5,
When the biometric sensor placed on the charging cradle is the second biometric sensor:
wherein the second biometric sensor configured to:
acquire the second information while being placed on the charging cradle; and
transmit the second information to the first biometric sensor via the second communication module,
wherein the first biometric sensor configured to:
acquire the first information while being attached to the wearable device;
receive the second information from the second biometric sensor via the first communication module; and
determine, via the biometric information processing module, overlapped information between the first information and the second information as first biometric information for the first region of the user.

8. The system of claim 2,
The wearable device further includes a camera module,
wherein the camera module is installed on the front portion of the wearable device with a field of view configured to capture the user's surrounding environment corresponding to the user's line of sight,
wherein the processor is configured to:
acquire movement information of the user via the IMU sensor;
acquire vision information corresponding to the user's line of sight via the camera module; and
transmit the movement information and the vision information to the external device via the third communication module,
wherein the external device is configured to:
determine at least one of the user's psychological state or stress level by inputting the second biometric information, the movement information, and the vision information into the artificial intelligence model.

9. The system of claim 1,
The wearable device further includes a GPS sensor, an illuminance sensor, and a microphone module,
wherein the processor is configured to:
acquire location information of the user via the GPS sensor;
acquire ambient brightness information via the illuminance sensor;
acquire ambient noise information via the microphone module;
receive weather information in real time from a server maintaining a weather information database via the third communication module; and
transmit the location information, the brightness information, the noise information, and the weather information to the external device via the third communication module,
wherein the external device is configured to:
determine at least one of the user's psychological state or stress level by inputting the second biometric information, the location information, the brightness information, the noise information, and the weather information into the artificial intelligence model.

10. The system of claim 1,
The wearable device includes:
a blower; and
a plurality of ventilation holes respectively formed on the front portion and the rear portion of the wearable device,
wherein the air tubes are disposed on the front portion and the rear portion of the wearable device, and the ventilation holes are positioned on the front portion and the rear portion such that they do not overlap with the air tubes,
wherein the processor is configured to:
measure a body temperature of the user via at least one of the first biometric sensor or the second biometric sensor; and
when the body temperature exceeds a reference value, operate the blower to supply air to the user wearing the wearable device until the body temperature falls below the reference value.
